(19) United States
(12) Patent Application Publication
(10) Pub. No.: US 2025/0352029 A1
(43) Pub. Date: Nov. 20, 2025

(54) MEDICAL DEVICE, MEDICAL SYSTEM, OPERATION METHOD OF MEDICAL DEVICE, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP)

(73) Assignee: OLYMPUS MEDICAL SYSTEMS CORP., Tokyo (JP)

(21) Appl. No.: 19/291,961

(22) Filed: Aug. 6, 2025

Related U.S. Application Data
(63) Continuation of application No. PCT/JP2023/004457, filed on Feb. 9, 2023.

(51) Int. Cl. A61B 1/00; A61B 1/04; A61B 1/06; G06T 7/00

(52) U.S. Cl. CPC A61B 1/000094; A61B 1/0005; A61B 1/043; A61B 1/0627; A61B 1/0661; G06T 7/0012; G06T 2207/10064; G06T 2207/10068; G06T 2207/30096; G06T 2207/30101

(57) ABSTRACT

A medical device includes a processor including hardware, the processor being configured to: acquire a display image in which a blood vessel region of a blood vessel in a subject is specified and a fluorescence image overlapping at least a part of a visual field region of the display image; specify thermal denaturation information in the blood vessel region based on the display image and the fluorescence image; and output the thermal denaturation information.

### CROSS REFERENCES TO RELATED APPLICATIONS

This application is a continuation of International Application No. PCT/JP2023/004457, filed on Feb. 9, 2023, the entire contents of which are incorporated herein by reference.

### BACKGROUND

### 1. Technical Field

The present disclosure relates to a medical device, a medical system, an operation method of the medical device, and a computer-readable recording medium.

### 2. Related Art

In the related art, in the medical field, a technique for visualizing a cauterization state of a subject such as a biological tissue using an energy device or the like is known (refer to, for example, WO 2020/054723 A). In this technique, a subject is irradiated with excitation light, and an image and information including fluorescence image data generated based on an imaging signal acquired by capturing fluorescence generated from a thermally invasive region of the subject by receiving the excitation light are displayed, thereby visualizing a cauterization state for a user such as an operator.

### SUMMARY

In some embodiments, a medical device includes a processor including hardware, the processor being configured to: acquire a display image in which a blood vessel region of a blood vessel in a subject is specified and a fluorescence image overlapping at least a part of a visual field region of the display image; specify thermal denaturation information in the blood vessel region based on the display image and the fluorescence image; and output the thermal denaturation information.

In some embodiments, a medical system includes: a light source device including a light source configured to emit excitation light for exciting an advanced glycation end-product generated by subjecting a biological tissue to a thermal treatment; an imaging device including an imaging element configured to generate an imaging signal by capturing fluorescence emitted by the excitation light; and a medical device including a processor including hardware, the processor being configured to: acquire, from the imaging element, a display image in which a blood vessel region of a blood vessel in a subject is specified and a fluorescence image overlapping at least a part of a visual field region of the display image; specify thermal denaturation information in the blood vessel region based on the display image and the fluorescence image; and output the thermal denaturation information.

In some embodiments, provided is an operation method of a medical device including a processor. The operation method causes the processor to execute: acquiring a display image in which a blood vessel region of a blood vessel in a subject is specified and a fluorescence image overlapping at least a part of a visual field region of the display image; specifying thermal denaturation information in the blood vessel region based on the display image and the fluorescence image; and outputting the thermal denaturation information.

In some embodiments, provided is a non-transitory computer-readable recording medium with an executable program stored thereon, the program causing a processor of a medical device driven to execute: acquiring a display image in which a blood vessel region of a blood vessel in a subject is specified and a fluorescence image overlapping at least a part of a visual field region of the display image; specifying thermal denaturation information in the blood vessel region based on the display image and the fluorescence image; and outputting the thermal denaturation information.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope system according to an embodiment;

FIG. 2 is a block diagram illustrating a functional configuration of a main part of the endoscope system according to the embodiment;

FIG. 3 is a diagram schematically illustrating wavelength characteristics of excitation light emitted by a light source unit according to the embodiment;

FIG. 4 is a diagram schematically illustrating a configuration of a pixel unit according to the embodiment;

FIG. 5 is a diagram schematically illustrating a configuration of a color filter according to the embodiment;

FIG. 6 is a diagram schematically illustrating sensitivity and a wavelength band of each filter according to the embodiment;

FIG. 7A is a diagram schematically illustrating signal values of G pixels of an imaging element according to the embodiment;

FIG. 7B is a diagram schematically illustrating signal values of R pixels of the imaging element according to the embodiment;

FIG. 7C is a diagram schematically illustrating signal values of B pixels of the imaging element according to the embodiment;

FIG. 8 is a diagram schematically illustrating a configuration of a cut filter according to the embodiment;

FIG. 9 is a diagram schematically illustrating transmission characteristics of the cut filter according to the embodiment;

FIG. 10 is a flowchart illustrating an outline of processing executed by a control device according to the embodiment;

FIG. 11 is a diagram illustrating an example of a white light image generated by a generation unit according to the embodiment;

FIG. 12 is a diagram schematically illustrating a detection result of detecting a blood vessel region in the white light image by a detector according to the embodiment;

FIG. 13 is a diagram schematically illustrating a correspondence relationship between a signal value of a fluorescence image, a degree of thermal denaturation, and a thermocoagulation level of a blood vessel according to the embodiment; and

FIG. 14 is a diagram illustrating an example of a synthetic image generated by a synthesis unit according to an embodiment.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to an extent that a content of the present disclosure can be understood. That is, the present disclosure is not limited only to the shape, the size, and the positional relationship illustrated in each drawing. Furthermore, in the description of the drawings, the same portions will be denoted by the same reference numerals. Furthermore, as an example of an endoscope system according to the present disclosure, an endoscope system including a rigid endoscope and a medical imaging device will be described.

### Configuration of Endoscope System

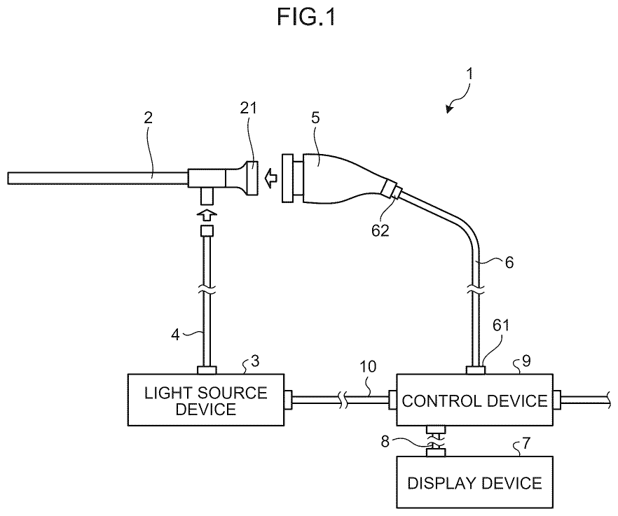

FIG. 1 is a diagram illustrating a schematic configuration of an endoscope system according to an embodiment. An endoscope system 1 illustrated in FIG. 1 is a system that is used in a medical field and observes and treats a biological tissue in a subject such as a living body. Note that, in the embodiment, a rigid endoscope system using a rigid endoscope (an insertion unit 2) illustrated in FIG. 1 will be described as the endoscope system 1, but the disclosure is not limited thereto, and for example, an endoscope system including a flexible endoscope may be used. Furthermore, the endoscope system 1 can also be applied to a medical microscope, a medical surgical robot system, or the like that includes a medical imaging device that captures a subject and performs surgery, processing, or the like while displaying an observation image based on an imaging signal (image data) captured by the medical imaging device on a display device. In addition, in recent years, in the medical field, minimally invasive treatment using an endoscope, a laparoscope, or the like has been widely performed. For example, as minimally invasive treatment using an endoscope, a laparoscope, or the like, endoscopic submucosal dissection (ESD), laparoscopy and endoscopy cooperative surgery (LECS), non-exposed endoscopic wall-inversion surgery (NEWS), transurethral resection of the bladder tumor (TUR-bt), or the like is widely performed. In the minimally invasive treatment, in the case of performing treatment, for example, in order to mark a region to be operated as pretreatment, an operator such as a doctor performs resection by cauterization, marking treatment by thermal treatment, or the like on a characteristic region (a pathogenic region) having an affected part with respect to a biological tissue using a treatment tool of an energy device that emits energy such as high frequency, ultrasonic, or microwave. In addition, in the case of actual treatment as well, the operator performs treatment such as resection and coagulation of a biological tissue of a subject using an energy device or the like. Therefore, the endoscope system 1 illustrated in FIG. 1 is used when the subject is operated or processed using a treatment tool (not illustrated) such as an energy device capable of performing thermal treatment.

Specifically, the endoscope system 1 illustrated in FIG. 1 is used for endoscopic submucosal dissection (ESD).

The endoscope system 1 illustrated in FIG. 1 includes an insertion unit 2, a light source device 3, a light guide 4, an endoscope camera head 5 (an endoscope imaging device), a first transmission cable 6, a display device 7, a second transmission cable 8, a control device 9, a third transmission cable 10, a perfusion device 11, and a fourth transmission cable 12.

The insertion unit 2 is rigid or at least partially flexible and has an elongated shape. The insertion unit 2 is inserted into a subject such as a patient via a trocar. The insertion unit 2 is provided with an optical system such as a lens that forms an observation image therein.

The light source device 3 is connected to one end of the light guide 4, and supplies illumination light to irradiate the inside of the subject to one end of the light guide 4 under the control of the control device 9. The light source device 3 is realized by using one or more light sources of a light emitting diode (LED) light source, a xenon lamp, and a semiconductor laser element such as a laser diode (LD), a processor, which is a processing device having hardware such as a field programmable gate array (FPGA) and a central processing unit (CPU), and a memory, which is a temporary storage area used by the processor. Note that the light source device 3 and the control device 9 may be configured to communicate individually as illustrated in FIG. 1, or may be integrated with each other.

One end of the light guide 4 is detachably connected to the light source device 3, and the other end thereof is detachably connected to the insertion unit 2. The light guide 4 guides illumination light supplied from the light source device 3 from one end to the other end and supplies the illumination light to the insertion unit 2.

An eyepiece unit 21 of the insertion unit 2 is detachably connected to the endoscope camera head 5. Under the control of the control device 9, the endoscope camera head 5 generates an imaging signal (RAW data) by receiving an observation image formed by the insertion unit 2 and performing photoelectric conversion, and outputs the imaging signal to the control device 9 via the first transmission cable 6.

One end of the first transmission cable 6 is detachably connected to the control device 9 via a video connector 61, and the other end thereof is detachably connected to the endoscope camera head 5 via a camera head connector 62. The first transmission cable 6 transmits the imaging signal output from the endoscope camera head 5 to the control device 9, and transmits setting data, power, and the like output from the control device 9 to the endoscope camera head 5. Here, the setting data is a control signal, a synchronization signal, a clock signal, and the like for controlling the endoscope camera head 5.

Under the control of the control device 9, the display device 7 displays an observation image based on an imaging signal subjected to image processing in the control device 9 and various types of information regarding the endoscope system 1. The display device 7 is realized by using a display monitor such as liquid crystal or organic electro luminescence (EL).

One end of the second transmission cable 8 is detachably connected to the display device 7, and the other end thereof is detachably connected to the control device 9. The second transmission cable 8 transmits the imaging signal subjected to the image processing in the control device 9 to the display device 7.

The control device 9 is realized by using a processor, which is a processing device having hardware such as a graphics processing unit (GPU), an FPGA, or a CPU, and a memory, which is a temporary storage area used by the processor. The control device 9 integrally controls operations of the light source device 3, the endoscope camera head 5, and the display device 7 via each of the first transmission cable 6, the second transmission cable 8, and the third transmission cable 10 according to a program recorded in the memory. In addition, the control device 9 performs various types of image processing on the imaging signal input via the first transmission cable 6 and outputs the imaging signal to the second transmission cable 8.

One end of the third transmission cable 10 is detachably connected to the light source device 3, and the other end thereof is detachably connected to the control device 9. The third transmission cable 10 transmits the control data from the control device 9 to the light source device 3.

### Functional Configuration of Main Part of Endoscope System

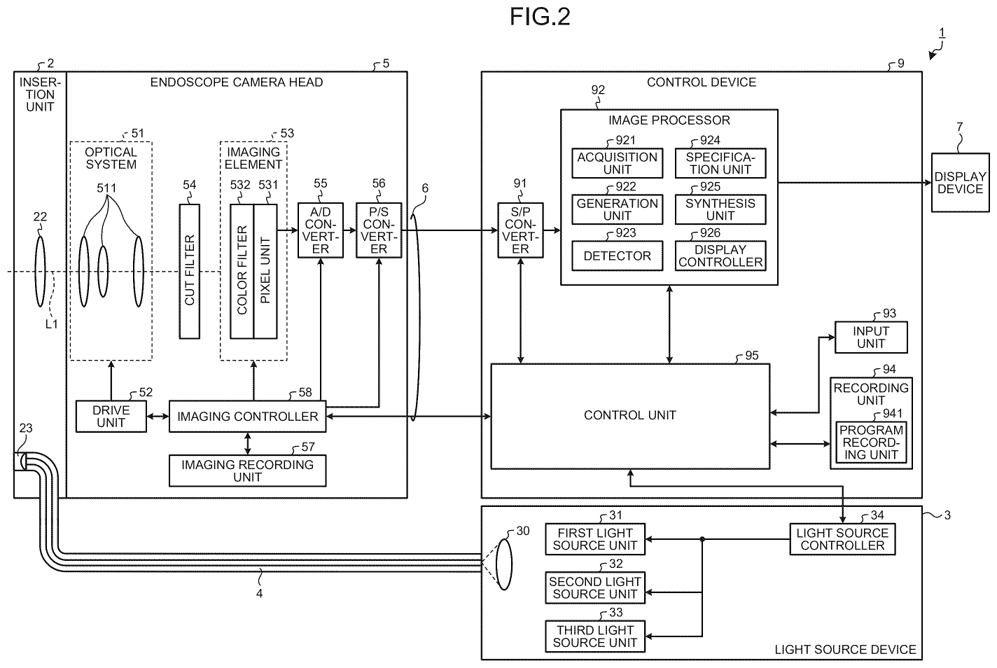

Next, a functional configuration of a main part of the above-described endoscope system 1 will be described. FIG. 2 is a block diagram illustrating a functional configuration of a main part of the endoscope system 1.

### Configuration of Insertion Unit

First, the configuration of the insertion unit 2 will be described. The insertion unit 2 includes an optical system 22 and an illumination optical system 23.

The optical system 22 forms a subject image by collecting light such as reflected light reflected from a subject, return light from the subject, excitation light from the subject, and fluorescence emitted from a thermally denatured region thermally denatured by thermal treatment of an energy device or the like. The optical system 22 is realized by using one or a plurality of lenses and the like.

The illumination optical system 23 irradiates the subject with illumination light supplied from the light guide 4. The illumination optical system 23 is realized by using one or a plurality of lenses or the like.

### Configuration of Light Source Device

Next, a configuration of the light source device 3 will be described. The light source device 3 includes a condenser lens 30, a first light source unit 31, a second light source unit 32, a third light source unit 33, and a light source controller 34.

The condenser lens 30 condenses light emitted by each of the first light source unit 31 and the third light source unit 33 and emits the light to the light guide 4.

Under the control of the light source controller 34, the first light source unit 31 supplies white light as illumination light to the light guide 4 by emitting white light (normal light) which is visible light. The first light source unit 31 includes a collimator lens, a white LED lamp, a drive driver, and the like. Note that the first light source unit 31 may supply visible white light by simultaneously emitting light using a red LED lamp, a green LED lamp, and a blue LED lamp. Of course, the first light source unit 31 may be configured using a halogen lamp, a xenon lamp, or the like.

Under the control of the light source controller 34, the second light source unit 32 emits first narrow band light having a predetermined wavelength band to supply the first narrow band light to the light guide 4 as illumination light. Here, the first narrow band light has a wavelength band ranging from 530 nm to 550 nm (a center wavelength is 540 nm). The second light source unit 32 includes a green LED lamp, a collimating lens, a transmission filter that transmits light ranging from 530 nm to 550 nm, a drive driver, and the like.

Under the control of the light source controller 34, the third light source unit 33 emits second narrow band light having a wavelength band different from that of the first narrow band light to supply the second narrow band light to the light guide 4 as illumination light. Here, the second narrow band light has a wavelength band ranging from 400 nm to 430 nm (a center wavelength is 415 nm). The third light source unit 33 is realized using a semiconductor laser such as a collimator lens or a violet laser diode (LD), a drive driver, and the like. Note that, in the embodiment, the second narrow band light functions as excitation light that excites advanced glycation end-products generated by subjecting a biological tissue to thermal treatment by an energy device or the like. When the amino acid and the reducing sugar are heated, a saccharification reaction (Maillard reaction) occurs. The end product resulting from this Maillard reaction is generally called advanced glycation end-products (AGEs). As characteristics of the AGEs, it is known that a substance having fluorescence characteristics is included. That is, when the biological tissue is thermally treated with an energy device, the AGEs are generated by heating the amino acid and the reducing sugar in the biological tissue to cause the Maillard reaction. The AGEs generated by this heating can visualize the state of thermal treatment by fluorescence observation. Furthermore, AGEs are known to emit stronger fluorescence than autofluorescent substances originally present in biological tissues. That is, in the embodiment, the thermally denatured region by the thermal treatment is visualized using the fluorescence characteristic of the AGEs generated in the biological tissue by the thermal treatment by the energy device or the like. Therefore, in the embodiment, the biological tissue is irradiated with excitation light of blue light having a wavelength of about 415 nm for exciting the AGEs from the third light source unit 33. As a result, in the embodiment, a fluorescence image (a thermally denatured image) can be observed based on an imaging signal obtained by capturing the fluorescence (for example, green light having a wavelength ranging from 490 to 625 nm) emitted from the thermally denatured region generated from the AGEs.

The light source controller 34 is realized by using a processor having hardware such as an FPGA or a CPU, and a memory, which is a temporary storage area used by the processor. The light source controller 34 controls light emission timing, light emission time, and the like of each of the first light source unit 31 and the third light source unit 33 based on control data input from the control device 9.

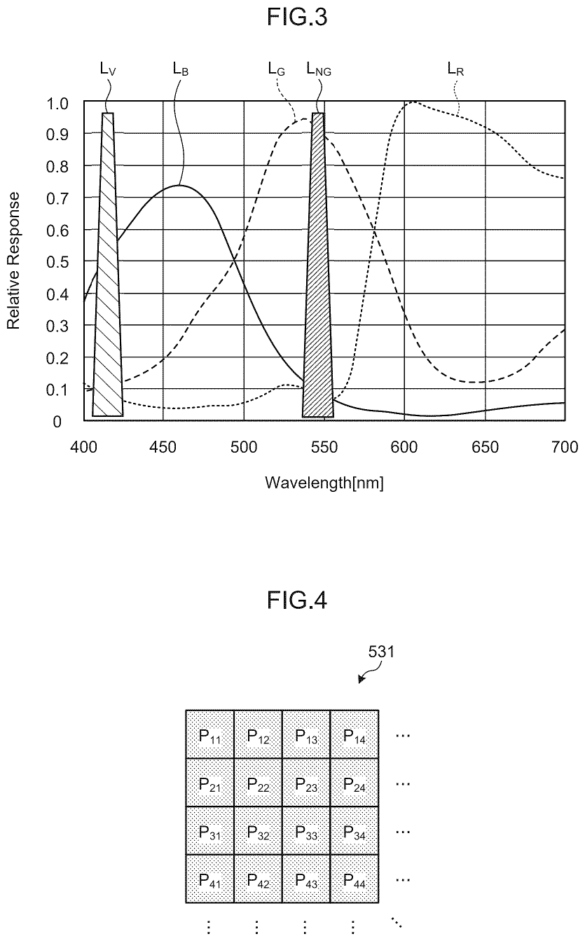

Here, wavelength characteristics of light emitted by the second light source unit 32 and the third light source unit 33 will be described. FIG. 3 is a diagram schematically illustrating wavelength characteristics of light emitted by each of the second light source unit 32 and the third light source unit 33. In FIG. 3, the horizontal axis represents a wavelength (nm), and the vertical axis represents wavelength characteristics. In FIG. 3, a polygonal line L_{NG} indicates a wavelength characteristic of the first narrow band light emitted by the second light source unit 32, and a polygonal line L_{V} indicates a wavelength characteristic of the second narrow band light (excitation light) emitted by the third light source unit 33. In FIG. 3, a curve L_{B} represents a blue wavelength band, a curve L_{G} represents a green wavelength band, and a curve L_{R} represents a red wavelength band.

As indicated by the polygonal line L_{NG} in FIG. 3, the second light source unit 32 emits narrow band light having a center wavelength (peak wavelength) of 540 nm and a wavelength band ranging from 530 nm to 550 nm. In addition, the third light source unit 33 emits excitation light having a center wavelength (peak wavelength) of 415 nm and a wavelength band ranging from 400 nm to 430 nm.

As described above, each of the second light source unit 32 and the third light source unit 33 emits the first narrow band light and the second narrow band light (excitation light) in different wavelength bands.

In addition, the first narrow band light is formed as light for layer determination in a biological tissue. Specifically, in the first narrow band light, a difference between absorbance of a mucosal layer, which is a subject, and absorbance of a muscle layer, which is a subject, is large enough to identify the two subjects. Therefore, in a second image for layer determination, acquired by irradiation of the first narrow band light for layer determination, a region in which the mucosal layer is captured has a smaller pixel value (a luminance value) and becomes darker compared to a region in which the muscle layer is captured. That is, in one embodiment, by using the second image for layer determination for generation of the display image, it is possible to set a display mode in which the mucosal layer and the muscle layer can be easily identified.

In addition, the second narrow band light (excitation light) is formed as light for layer determination in a biological tissue, which is different from the first narrow band light. Specifically, in the second narrow band light, a difference between absorbance of a muscle layer, which is a subject, and absorbance of a fat layer, which is a subject, is large enough to identify the two subjects. Therefore, in the second image for layer determination, acquired by irradiation of the second narrow band light for layer determination, a region in which the muscle layer is captured has a smaller pixel value (a luminance value) and becomes darker compared to a region in which the fat layer is captured. That is, by using the second image for layer determination for generation of the display image, it is possible to set an aspect in which the muscle layer and the fat layer are easily identified.

Both the mucosal layer (biological mucosa) and the muscle layer are subjects containing a large amount of myoglobin. However, the concentration of myoglobin contained is relatively high in the mucosal layer and is relatively low in the muscle layer. A cause of a difference in light absorption characteristics between the mucosal layer and the muscle layer is due to a difference in the concentration of myoglobin contained in each of the mucosal layer (biological mucosa) and the muscle layer. A difference in absorbance between the mucosal layer and the muscle layer becomes maximum in the vicinity of a wavelength at which the absorbance of the biological mucosa becomes the maximum value. That is, the first narrow band light for layer determination is light in which a difference between the mucosal layer and the muscle layer appears larger than light having a peak wavelength in another wavelength band.

In addition, since the second narrow band light has a lower absorbance of fat than an absorbance of the muscle layer, in the second image captured by emission of the second narrow band light for layer determination, a pixel value (luminance value) of a region in which the muscle layer is captured is smaller than a pixel value (luminance value) of a region in which the fat layer is captured. In particular, since the second narrow band light for layer determination is light corresponding to a wavelength at which the absorbance of the muscle layer becomes the maximum value, the second narrow band light becomes light in which a difference between the muscle layer and the fat layer significantly appears. That is, a difference between the pixel value (luminance value) of the muscle layer region and the pixel value (luminance value) of the fat layer region in the second image for layer determination is increased to an identifiable extent.

As described above, the light source device 3 irradiates the biological tissue with each of the first narrow band light and the second narrow band light. As a result, the endoscope camera head 5 to be described later can identify each of the mucosal layer, the muscle layer, and the fat layer constituting the biological tissue by capturing return light from the biological tissue, and can obtain an image capable of specifying a blood vessel region.

### Configuration of Endoscope Camera Head

Referring back to FIG. 2, the description of the configuration of the endoscope system 1 will be continued. Next, a configuration of the endoscope camera head 5 will be described. The endoscope camera head 5 includes an optical system 51, a drive unit 52, an imaging element 53, a cut filter 54, an A/D converter 55, a P/S converter 56, an imaging recording unit 57, and an imaging controller 58.

The optical system 51 forms a subject image collected by the optical system 22 of the insertion unit 2 on the light receiving surface of the imaging element 53. The optical system 51 can change the focal length and the focal position. The optical system 51 includes a plurality of lenses 511. The optical system 51 changes the focal length and the focal position by moving each of the plurality of lenses 511 on an optical axis L1 by the drive unit 52.

Under the control of the imaging controller 58, the drive unit 52 moves the plurality of lenses 511 of the optical system 51 along the optical axis L1. The drive unit 52 includes motors such as a stepping motor, a DC motor, and a voice coil motor, and a transmission mechanism such as a gear that transmits rotation of the motor to the optical system 51.

The imaging element 53 is implemented by using a charge coupled device (CCD) or complementary metal oxide semiconductor (CMOS) image sensor having a plurality of pixels arranged in a two-dimensional matrix. Under the control of the imaging controller 58, the imaging element 53 receives a subject image (light beam) that is formed by the optical system 51 and passes through the cut filter 54, performs photoelectric conversion, generates an imaging signal (RAW data), and outputs the imaging signal to the A/D converter 55. The imaging element 53 includes a pixel unit 531 and a color filter 532.

FIG. 4 is a diagram schematically illustrating a configuration of the pixel unit 531. As illustrated in FIG. 4, in the pixel unit 531, a plurality of pixels Pₙₘ (n=integer greater than or equal to 1, m=integer greater than or equal to 1) such as photodiodes that accumulate charges according to the amount of light are arranged in a two-dimensional matrix. Under the control of the imaging controller 58, the pixel unit 531 reads an image signal as image data from a pixel Pₙₘ in a reading region arbitrarily set as a reading target among the plurality of pixels Pₙₘ, and outputs the image signal to the A/D converter 55.

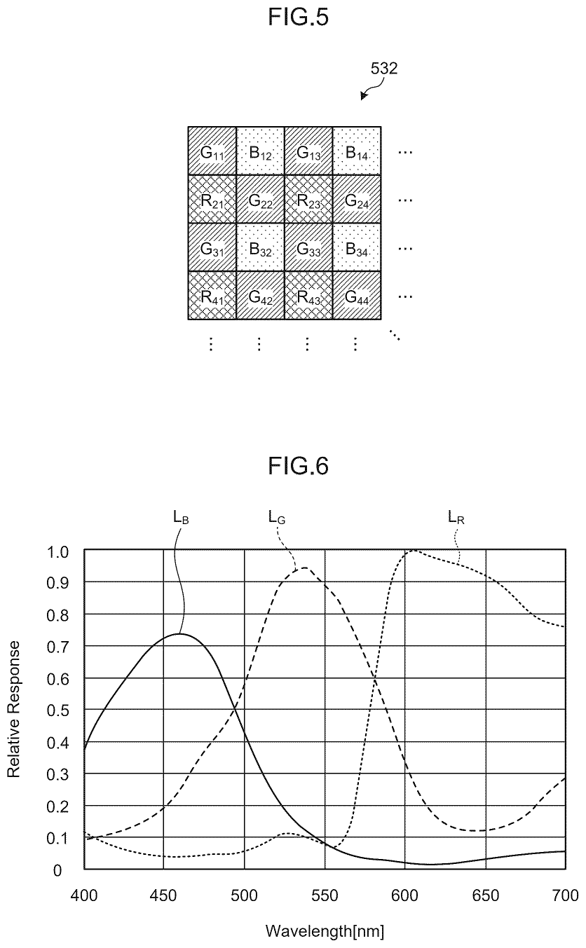

FIG. 5 is a diagram schematically illustrating a configuration of the color filter 532. As illustrated in FIG. 5, the color filter 532 includes a Bayer array having 2×2 as one unit. The color filter 532 includes a filter R that transmits light in a red wavelength band, two filters G that transmit light in a green wavelength band, and a filter B that transmits light in a blue wavelength band.

FIG. 6 is a diagram schematically illustrating sensitivity and a wavelength band of each filter. In FIG. 6, the horizontal axis represents a wavelength (nm), and the vertical axis represents transmission characteristics (sensitivity characteristics). In FIG. 6, a curve L_{B} represents the transmission characteristics of the filter B, a curve L_{G} represents the transmission characteristic of the filter G, and a curve L_{R} represents the transmission characteristic of the filter R.

As indicated by the curve L_{B} in FIG. 6, the filter B transmits light in a blue wavelength band. As indicated by the curve L_{G} in FIG. 6, the filter G transmits light in a green wavelength band. Further, as indicated by the curve L_{R} in FIG. 6, the filter R transmits light in a red wavelength band. Note that, in the following description, a pixel Pom in which the filter R is arranged on the light receiving surface will be described as an R pixel, a pixel Pom in which the filter G is arranged on the light receiving surface will be described as a G pixel, and a pixel Pₙₘ in which the filter B is arranged on the light receiving surface will be described as a B pixel.

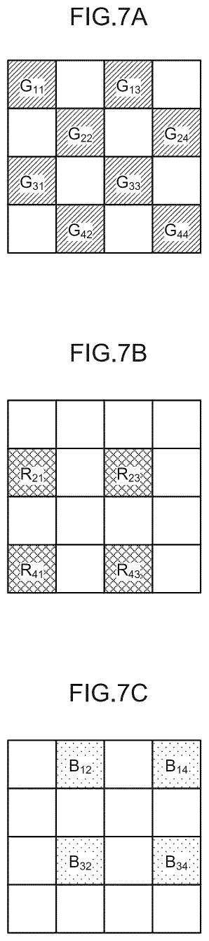

According to the imaging element 53 configured as described above, in a case where the subject image formed by the optical system 51 is received, a color signal (R signal, G signal, and B signal) of each of the R pixel, the G pixel, and the B pixel is generated (refer to FIGS. 7A to 7C).

Referring back to FIG. 2, the description of the configuration of the endoscope system 1 will be continued.

The cut filter 54 is disposed on the optical axis L1 between the optical system 51 and the imaging element 53. The cut filter 54 is provided on the light receiving surface side (incident surface side) of the G pixel provided with the filter G that transmits at least the green wavelength band of the color filter 532. The cut filter 54 shields light in a wavelength band of a short wavelength including a wavelength band of excitation light, and transmits a wavelength band on a longer wavelength side than the wavelength band of the excitation light.

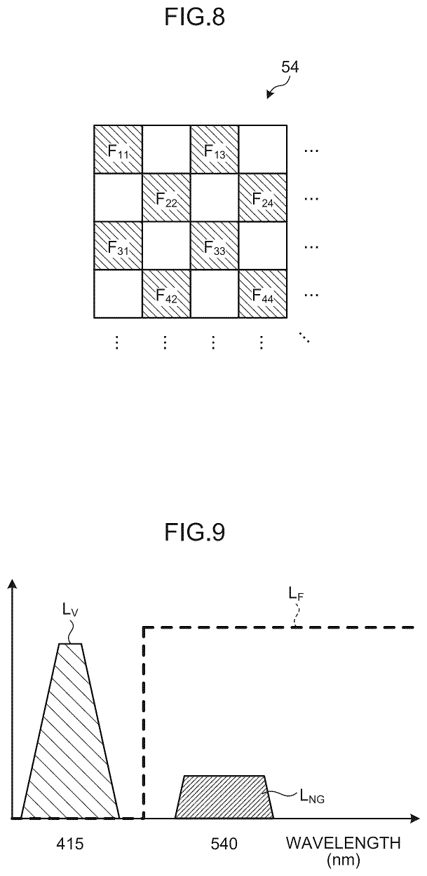

FIG. 8 is a diagram schematically illustrating a configuration of the cut filter 54. As illustrated in FIG. 8, a filter F₁₁ constituting the cut filter 54 is arranged at a position where the filter Gu (refer to FIG. 5) is arranged, and is arranged on the light receiving surface side directly above the filter Gn.

FIG. 9 is a diagram schematically illustrating transmission characteristics of the cut filter 54. In FIG. 8, the horizontal axis represents a wavelength (nm), and the vertical axis represents transmission characteristics. In FIG. 8, a polygonal line L_{F} represents a transmission characteristic of the cut filter 54, a polygonal line L_{NG} represents a wavelength characteristic of fluorescence, and a polygonal line L_{V} represents a wavelength characteristic of excitation light.

As illustrated in FIG. 9, the cut filter 54 shields a wavelength band of an excitation light and transmits a wavelength band on a long wavelength side from the wavelength band of the excitation light. Specifically, the cut filter 54 shields light in a wavelength band on a short wavelength side of 400 nm to less than 430 nm including the wavelength band of the excitation light, and transmits light in a wavelength band on a longer wavelength side than 400 nm to 430 nm including the excitation light.

Returning to FIG. 2, the description of the configuration of the endoscope camera head 5 will be continued.

Under the control of the imaging controller 58, the A/D converter 55 performs A/D conversion processing on an analog imaging signal input from the imaging element 53, and outputs the analog imaging signal to the P/S converter 56. The A/D converter 55 is implemented by using an A/D conversion circuit or the like.

Under the control of the imaging controller 58, the P/S converter 56 performs parallel/serial conversion on a digital imaging signal input from the A/D converter 55, and outputs the imaging signal subjected to the parallel/serial conversion to the control device 9 via the first transmission cable 6. The P/S converter 56 is implemented by using a P/S conversion circuit or the like. Note that, in the first embodiment, an E/O converter that converts an imaging signal into an optical signal may be provided instead of the P/S converter 56, and the imaging signal may be output to the control device 9 by the optical signal, or the imaging signal may be transmitted to the control device 9 by, for example, wireless communication such as Wireless Fidelity (Wi-Fi) (registered trademark).

The imaging recording unit 57 records various types of information (for example, pixel information of the imaging element 53 and characteristics of the cut filter 54) regarding the endoscope camera head 5. Furthermore, the imaging recording unit 57 records various setting data and control parameters transmitted from the control device 9 via the first transmission cable 6. The imaging recording unit 57 is configured using a nonvolatile memory or a volatile memory.

The imaging controller 58 controls the operation of each of the drive unit 52, the imaging element 53, the A/D converter 55, and the P/S converter 56 based on the setting data received from the control device 9 via the first transmission cable 6. The imaging controller 58 is realized by using a timing generator (TG), a processor having hardware such as an application specific integrated circuit (ASIC) or a CPU, and a memory, which is a temporary storage area used by the processor.

### Configuration of Control Device

Next, a configuration of the control device 9 will be described.

The control device 9 includes an S/P converter 91, an image processor 92, an input unit 93, a recording unit 94, and a control unit 95.

Under the control of the control unit 95, the S/P converter 91 performs serial/parallel conversion on image data received from the endoscope camera head 5 via the first transmission cable 6 and outputs the image data to the image processor 92. Note that, in a case where the endoscope camera head 5 outputs an imaging signal as an optical signal, an O/E converter that converts an optical signal into an electric signal may be provided instead of the S/P converter 91. Furthermore, in a case where the endoscope camera head 5 transmits an imaging signal by wireless communication, a communication module capable of receiving a wireless signal may be provided instead of the S/P converter 91.

Under the control of the control unit 95, the image processor 92 performs predetermined image processing on an imaging signal of parallel data input from the S/P converter 91 and outputs the imaging signal to the display device 7. Here, the predetermined image processing is demosaic processing, white balance processing, gain adjustment processing, y correction processing, format conversion processing, and the like. The image processor 92 is implemented by using a processor which is a processing device having hardware such as a GPU or an FPGA and a memory which is a temporary storage area used by the processor. Specifically, the image processor 92 includes an acquisition unit 921, a generation unit 922, a detector 923, a specification unit 924, a synthesis unit 925, and a display controller 926.

The acquisition unit 921 acquires an imaging signal generated by capturing an image by the endoscope camera head 5 via the insertion unit 2. Specifically, when the light source device 3 emits any one of white light, narrow band light, and excitation light toward a biological tissue, the acquisition unit 921 acquires an imaging signal generated by capturing an image by the imaging element 53 of the endoscope camera head 5 from the endoscope camera head 5 via the S/P converter 91.

The generation unit 922 generates a white light image, a blood vessel recognition image of a special light image, and a fluorescence image based on the imaging signal acquired by the acquisition unit 921. Specifically, the generation unit 922 performs demosaic processing, white balance processing, gain adjustment processing, y correction processing, and the like on the imaging signal acquired by the acquisition unit 921 to generate the white light image. More specifically, in a case where the light source device 3 emits white light toward the biological tissue, the generation unit 922 generates the white light image by performing demosaic processing or the like on the imaging signal acquired by the acquisition unit 921. In addition, when the light source device 3 emits the narrow band light toward the biological tissue, the generation unit 922 performs image processing on the signal values of the G pixel and the B pixel included in the imaging signal acquired by the acquisition unit 921 to generate a blood vessel recognition image which is a pseudo color image (a narrow band image which is one of special light images). Furthermore, in a case where the light source device 3 irradiates the biological tissue with the excitation light, the generation unit 922 generates a fluorescence image by performing demosaic processing or the like on the imaging signal acquired by the acquisition unit 921.

The detector 923 detects a blood vessel region in the white light image based on the white light image and the blood vessel recognition image generated by the generation unit 922. Specifically, the detector 923 detects the blood vessel region in the white light image based on feature data of the blood vessel recognition image generated by the generation unit 922. For example, the detector 923 extracts the feature data by performing binarization processing, edge extraction processing, or the like of a well-known technique on the blood vessel recognition image, and detects the blood vessel region in the white light image based on the extracted feature data. The detector 923 detects the blood vessel region in the white light image by performing binarization processing or the like based on the feature data of the blood vessel recognition image, but the disclosure is not limited thereto. For example, the blood vessel region in the white light image may be detected using a learned model in which a plurality of blood vessel recognition images are learned by machine learning such as deep learning. In this case, the learned model learns, as input parameters, a plurality of blood vessel recognition images and training data (learning data) in which annotation or the like in which position designation such as a pixel address is performed on a blood vessel region included in each of the plurality of blood vessel recognition images is performed, and detects and outputs the position of the blood vessel region in the white light image as an output parameter (learning result). That is, the detector 923 may be configured to input the white light image and the blood vessel recognition image as the input parameters using the above-described learned model, and to output the position of the blood vessel region in the white light image as the output parameter.

The specification unit 924 specifies thermal denaturation information in the blood vessel region of the white light image based on the blood vessel region of the white light image detected by the detector 923 and the fluorescence image generated by the generation unit 922. Specifically, the specification unit 924 specifies the thermal denaturation information based on the signal value of each pixel of a region of the fluorescence image corresponding to the blood vessel region of the white light image.

The synthesis unit 925 generates a synthetic image obtained by synthesizing the thermal denaturation information specified by the specification unit 924 with the blood vessel region of the white light image detected by the detector 923.

The display controller 926 outputs various types of information regarding the endoscope system 1 to the display device 7 under the control of the control unit 95. In addition, the display controller 926 outputs the synthetic image generated by the synthesis unit 925 to the display device 7.

The input unit 93 receives inputs of various operations related to the endoscope system 1 and outputs the received operations to the control unit 95. The input unit 93 includes a mouse, a foot switch, a keyboard, a button, a switch, a touch panel, and the like.

The recording unit 94 is implemented by using a recording medium such as a volatile memory, a nonvolatile memory, a solid state drive (SSD), a hard disk drive (HDD), or a memory card. The recording unit 94 records data including various parameters and the like necessary for the operation of the endoscope system 1. Furthermore, the recording unit 94 includes a program recording unit 941 that records various programs for operating the endoscope system 1.

The control unit 95 is realized by using a processor having hardware such as an FPGA or a CPU, and a memory, which is a temporary storage area used by the processor. The control unit 95 integrally controls each of the units constituting the endoscope system 1.

Specifically, the control unit 95 reads and executes a program recorded in the program recording unit 941 in a work area of a memory, and controls each component and the like through execution of the program by the processor, so that hardware and software cooperate with each other to realize a functional module matching a predetermined purpose.

### Processing of Control Device

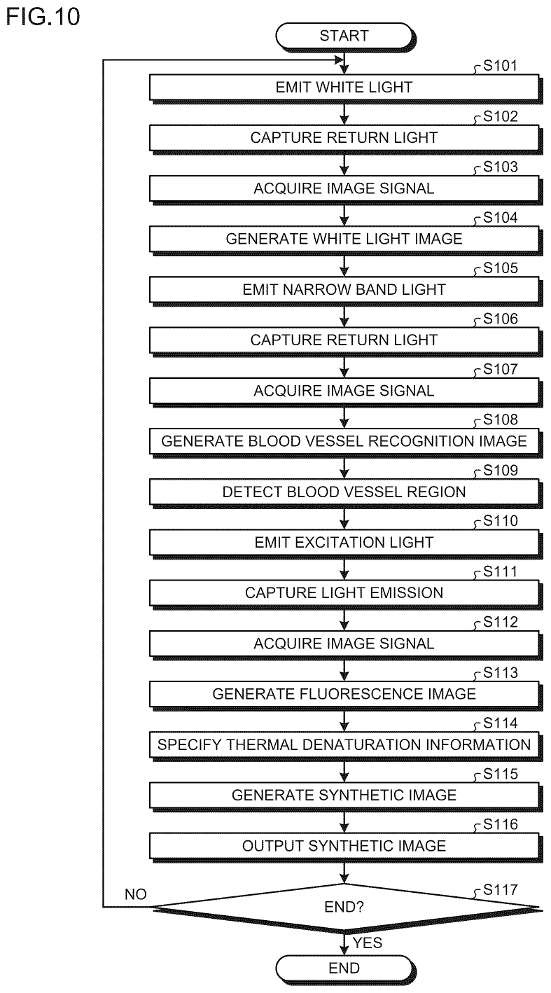

Next, processing executed by the control device 9 will be described. FIG. 10 is a flowchart illustrating an outline of processing executed by the control device 9.

First, as illustrated in FIG. 10, the control unit 95 causes the first light source unit 31 of the light source device 3 to emit light and supplies white light to the insertion unit 2 to irradiate the biological tissue with white light (Step S101).

Subsequently, the control unit 95 causes the imaging element 53 of the endoscope camera head 5 to capture return light from the biological tissue and reflected light from the biological tissue (Step S102).

Thereafter, the acquisition unit 921 acquires an imaging signal generated by capturing an image using the imaging element 53 of the endoscope camera head 5 (Step S103).

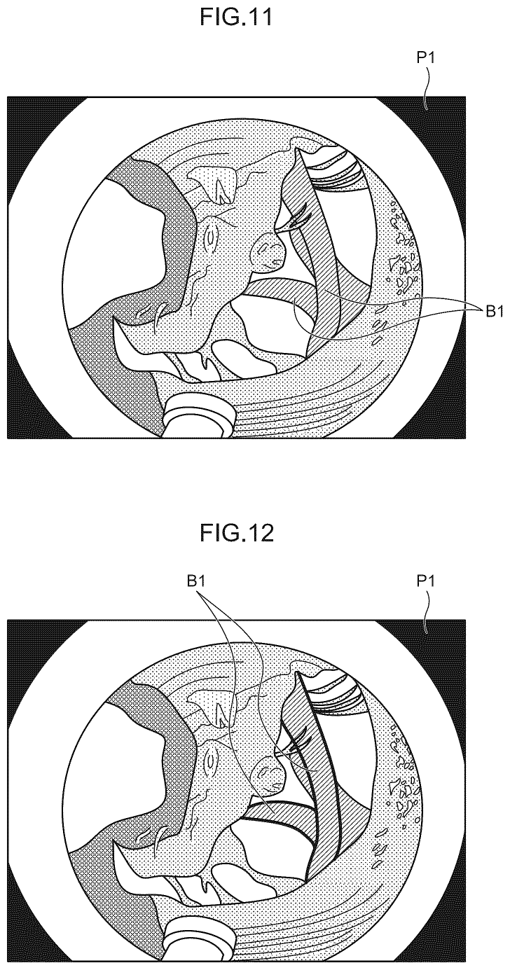

Subsequently, the generation unit 922 generates a white light image in a subject based on the imaging signal acquired by the acquisition unit 921 (Step S104). FIG. 11 is a diagram illustrating an example of the white light image generated by the generation unit 922. As illustrated in FIG. 11, the generation unit 922 performs demosaic processing, white balance processing, gain adjustment processing, y correction processing, and the like on the imaging signal acquired by the acquisition unit 921 to generate a white light image P1 including a blood vessel region B1 of a blood vessel in the biological tissue in the subject.

Thereafter, the control unit 95 causes each of the second light source unit 32 and the third light source unit 33 of the light source device 3 to emit light and supplies narrow band light (first narrow band light and second narrow band light) to the insertion unit 2, thereby emitting the narrow band light for blood vessel recognition in the biological tissue (Step S105).

Subsequently, the control unit 95 causes the imaging element 53 of the endoscope camera head 5 to capture return light from the biological tissue and reflected light from the biological tissue (Step S106).

Thereafter, the acquisition unit 921 acquires an imaging signal from the imaging element 53 of the endoscope camera head 5 (Step S107).

Subsequently, the generation unit 922 generates a blood vessel recognition image based on the imaging signal acquired by the acquisition unit 921 (Step S108). Specifically, the generation unit 922 performs image processing on signal values of a G pixel and a B pixel included in the imaging signal acquired by the acquisition unit 921 to generate the blood vessel recognition image which is a pseudo color image (narrow band image). In this case, the signal value of the G pixel includes mucosal deep layer information of the subject. Furthermore, the signal value of the B pixel includes mucosal surface layer information of the subject. Therefore, the generation unit 922 performs image processing such as gain control processing, pixel complement processing, and mucosal enhancement processing on the signal value of each of the G pixel and the B pixel included in the imaging signal to generate the blood vessel recognition image which is the pseudo color image. Here, the blood vessel recognition image is an image generated using only the signal value of the G pixel and the signal value of the B pixel. Note that the generation unit 922 deletes the signal value of the R pixel included in the imaging signal acquired by the acquisition unit 921 without using the signal value for generation of the blood vessel recognition image.

Thereafter, the detector 923 detects a blood vessel region in the white light image based on the white light image and the blood vessel recognition image generated by the generation unit 922 (Step S109). FIG. 12 is a diagram schematically illustrating a detection result of detecting the blood vessel region in the white light image by the detector 923. As illustrated in FIG. 12, the detector 923 detects the blood vessel region B1 on the white light image P1 based on feature data of the blood vessel recognition image generated by the generation unit 922. For example, the detector 923 extracts the feature data by performing binarization processing, edge extraction processing, or the like of a well-known technique on the blood vessel recognition image, and detects the blood vessel region B1 in the white light image P1 based on the extracted feature data. In this case, the detector 923 detects a region of a pixel address of the white light image P1, corresponding to a pixel address of the blood vessel region B1 detected in the blood vessel recognition image, as the blood vessel region B1 of the white light image P1.

Subsequently, the control unit 95 causes the third light source unit 33 of the light source device 3 to emit light and supplies excitation light to the insertion unit 2, thereby irradiating the biological tissue with the excitation light for emitting the thermally denatured region (Step S110).

Thereafter, the control unit 95 causes the imaging element 53 of the endoscope camera head 5 to capture light emission from the biological tissue (Step S111).

Subsequently, the acquisition unit 921 acquires an imaging signal from the imaging element 53 of the endoscope camera head 5 (Step S112).

Thereafter, the generation unit 922 generates a fluorescence image based on the imaging signal acquired by the acquisition unit 921 (Step S113).

Subsequently, the specification unit 924 specifies thermal denaturation information in the blood vessel region of the white light image based on the blood vessel region B1 of the white light image P1 detected by the detector 923 and the fluorescence image generated by the generation unit 922 (Step S114). Specifically, the specification unit 924 specifies the thermal denaturation information based on the signal value of each pixel of a region of the fluorescence image corresponding to the blood vessel region B1 of the white light image P1.

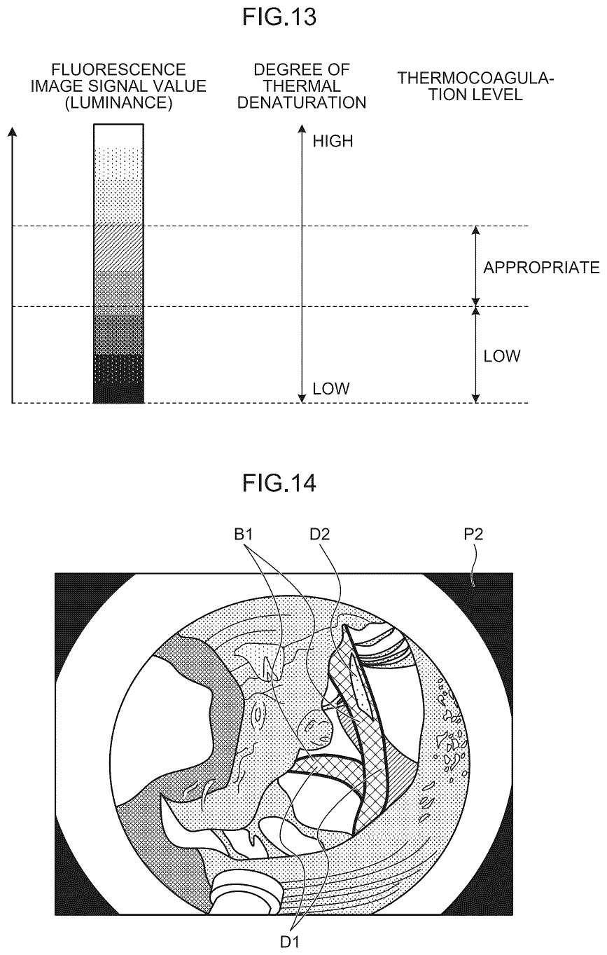

FIG. 13 is a diagram schematically illustrating a correspondence relationship among a signal value of a fluorescence image, a degree of thermal denaturation, and a thermocoagulation level of a blood vessel. As illustrated in FIG. 13, the specification unit 924 specifies a region having a low thermocoagulation level as the thermal denaturation information based on the signal value of each pixel in the region of the fluorescence image corresponding to the blood vessel region B1 of the white light image P1. Specifically, the specification unit 924 determines whether the signal value is smaller than a predetermined value for each pixel in the region of the fluorescence image corresponding to the blood vessel region B1 of the white light image P1. Then, the specification unit 924 specifies, as a region having a low thermocoagulation level, a region of a pixel, the signal value of which is smaller than the predetermined value in the region of the fluorescence image corresponding to the blood vessel region B1 of the white light image P1. On the other hand, the specification unit 924 specifies, as a region having a high thermocoagulation level, a region of a pixel, the signal value of which is not smaller than the predetermined value in the region of the fluorescence image corresponding to the blood vessel region B1 of the white light image P1, that is, a region of a pixel having a signal value equal to or larger than the predetermined value.

Thereafter, the synthesis unit 925 generates a synthetic image in which the thermal denaturation information specified by the specification unit 924 is synthesized with the blood vessel region B1 of the white light image P1 detected by the detector 923 (Step S115). FIG. 14 is a diagram illustrating an example of a synthetic image generated by the synthesis unit 925. As illustrated in FIG. 14, the synthesis unit 925 generates a synthetic image P2 obtained by synthesizing thermal denaturation information D1 and D2 specified by the specification unit 924 with the blood vessel region B1 of the white light image P1 detected by the detector 923. In this case, the synthesis unit 925 synthesizes the outline of the blood vessel region B1 of the white light image P1 detected by the detector 923 with the synthetic image P2. Here, the thermal denaturation information D1 is a region where the thermocoagulation level is higher than or equal to a predetermined value by the specification unit 924. Further, the thermal denaturation information D2 is a region where the thermocoagulation level is lower than the predetermined value by the specification unit 924. In FIG. 14, the thermal denaturation information D1 and the thermal denaturation information D2 are indicated by different hatchings in order to distinguish the thermal denaturation information D1 and D2. That is, the synthesis unit 925 synthesizes the thermal denaturation information D1 and the thermal denaturation information D2 in colors different from each other so as to be distinguishable from the blood vessel region B1 of the white light image P1 detected by the detector 923, thereby generating the synthetic image P2.

Subsequently, the display controller 926 outputs the synthetic image P2 generated by the synthesis unit 925 to the display device 7 (Step S116). As a result, an operator can intuitively grasp the thermocoagulation level in the blood vessel region B1 without relying on his/her empirical rule.

Thereafter, the control unit 95 determines whether an end signal for ending the observation of the subject by the endoscope system 1 is input from the input unit 93 (Step S117). When the control unit 95 determines that the end signal for ending the observation of the subject by the endoscope system 1 is input from the input unit 93 (Step S117: Yes), the endoscope system 1 ends this processing. On the other hand, when the control unit 95 determines that the end signal for ending the observation of the subject by the endoscope system 1 is not input from the input unit 93 (Step S117: No), the endoscope system 1 returns to Step S101 described above.

According to one embodiment described above, since the display controller 926 outputs the thermal denaturation information in the blood vessel region B1 of the white light image P1 specified by the specification unit 924 to the display device 7, the operator can grasp the state of the coagulation denaturation to the blood vessel.

Furthermore, according to one embodiment, since the display controller 926 outputs the synthetic image P2 generated by the synthesis unit 925 to the display device 7, the operator can intuitively grasp the state of coagulation denaturation in the blood vessel.

Furthermore, according to one embodiment, since the specification unit 924 specifies the thermal denaturation information based on the signal value of each pixel constituting the fluorescence image, it is possible to accurately specify a region in which the thermal denaturation has occurred.

In addition, according to one embodiment, since the specification unit 924 specifies a region of a pixel in which a signal value is smaller than the predetermined value in a region of the fluorescence image corresponding to the blood vessel region B1 of the white light image P1 as a region having the low thermocoagulation level, it is possible to identify and specify the region having the high thermocoagulation level and the region having the low thermocoagulation level.

In addition, according to one embodiment, since the specification unit 924 specifies the thermocoagulation level in the blood vessel region B1 of the white light image P1, it is possible to present the state of thermal denaturation for a blood vessel region most desired by the operator.

Note that, in one embodiment, the display controller 926 may superimpose thermal denaturation information on the blood vessel region B1 of the white light image P1 specified by the specification unit 924 and output the thermal denaturation information superimposed on the blood vessel region B1 to the display device 7.

Furthermore, in one embodiment, the detector 923 may specify the blood vessel region from the feature data of the white light image generated by the generation unit 922. For example, the detector 923 may extract the feature data by performing binarization processing, edge extraction processing, or the like of a well-known technology on a signal value of a specific component, for example, the G pixel, for the white light image, and detect a blood vessel region in the white light image based on the extracted feature data.

Furthermore, in one embodiment, as the special light observation, the narrow band light observation (NBI) of irradiating the biological tissue with the narrow band light including the first narrow band light (530 nm to 550 nm) and the second narrow band light (400 nm to 430 nm) is used, but the disclosure is not limited thereto, and for example, other special light observation can also be applied. For example, as the special light observation, it is also possible to apply red light observation (RDI: Red Dichromatic Imaging) in which a biological tissue is irradiated with light in a green wavelength band and special light including light in an amber wavelength band and light in a red wavelength band, and a blood vessel and a bleeding portion in a deep portion such as mucosa can be grasped. In this case, the detector 923 detects the blood vessel region from the blood vessel recognition image of the red light observation image. This makes it possible to easily detect a blood vessel in a deep portion such as a mucosa.

### OTHER EMBODIMENTS

Various embodiments can be formed by appropriately combining a plurality of components disclosed in the endoscope system according to the above-described embodiment of the present disclosure. For example, some components may be deleted from all the components described in the endoscope system according to the above-described embodiment of the present disclosure. Furthermore, the components described in the endoscope system according to the above-described embodiment of the present disclosure may be appropriately combined with each other.

Furthermore, in the endoscope system according to the embodiment of the present disclosure, the endoscope systems are connected to each other by wire, but may be wirelessly connected to each other via a network.

Furthermore, in one embodiment of the present disclosure, the functions of the image processor 92 provided in the endoscope system, that is, the functional modules of the acquisition unit 921, the generation unit 922, the detector 923, the specification unit 924, the synthesis unit 925, and the display controller 926 may be provided in a server or the like connectable via a network. Of course, a server may be provided for each functional module.

In addition, in one embodiment of the present disclosure, an example of being used for transurethral bladder tumor resection has been described, but the present disclosure is not limited thereto, and can be applied to various treatments of resection of a lesion by, for example, an energy device or the like.

Furthermore, in the endoscope system according to one embodiment of the present disclosure, the above-described “unit” can be replaced with “means”, “circuit”, or the like. For example, the control unit can be replaced with a control means or a control circuit.

Note that, in the description of the flowcharts in the present specification, the context of processing between steps is clearly indicated using expressions such as “first”, “thereafter”, and “subsequently”, but the order of processing necessary for implementing the embodiments is not uniquely determined by such expressions. That is, the order of processing in the flowcharts described in the present specification can be changed within a range without inconsistency.

According to the present disclosure, there is an effect that the state of coagulation denaturation in a blood vessel can be confirmed.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising a processor comprising hardware, the processor being configured to:
acquire a display image in which a blood vessel region of a blood vessel in a subject is specified and a fluorescence image overlapping at least a part of a visual field region of the display image;
specify thermal denaturation information in the blood vessel region based on the display image and the fluorescence image; and
output the thermal denaturation information.

2. The medical device according to claim 1, wherein the processor is further configured to:
generate a synthetic image obtained by synthesizing the thermal denaturation information with the blood vessel region of the display image; and
output the synthetic image.

3. The medical device according to claim 1, wherein
the processor is further configured to superimpose the thermal denaturation information on the blood vessel region of the display image and output the thermal denaturation information superimposed on the blood vessel region.

4. The medical device according to claim 1, wherein the processor is further configured to:
acquire an imaging signal obtained by capturing fluorescence emitted from a thermally denatured region; and
generate the fluorescence image based on the imaging signal.

5. The medical device according to claim 4, wherein
the fluorescence is emitted from an advanced glycation end-product generated by subjecting a biological tissue to a thermal treatment.

6. The medical device according to claim 1, wherein
the processor is further configured to specify the thermal denaturation information based on a signal value of each pixel forming the fluorescence image.

7. The medical device according to claim 6, wherein
the processor is further configured to specify a region in which a thermocoagulation level is low as the thermal denaturation information based on the signal value of each pixel forming the fluorescence image.

8. The medical device according to claim 7, wherein the processor is further configured to:
determine whether the signal value is smaller than a predetermined value for each pixel forming the fluorescence image; and
specify a region of a pixel in which the signal value is smaller than the predetermined value as the region in which the thermocoagulation level is low.

9. The medical device according to claim 8, wherein
the processor is further configured to specify the thermocoagulation level in the blood vessel region.

10. The medical device according to claim 9, wherein
the processor is further configured to output the region in which the thermocoagulation level is lower than the predetermined value in a distinguishable manner as compared with a region in which the thermocoagulation level is higher than or equal to the predetermined value.

11. The medical device according to claim 1, wherein
the display image is an image in which the blood vessel region is specified from feature data in a white light image.

12. The medical device according to claim 1, wherein
the display image is an image in which the blood vessel region is specified from feature data in a special light image.

13. The medical device according to claim 1, wherein
the processor is further configured to generate the display image based on a white light image and a blood vessel recognition image having a visual field region identical to a visual field region of the white light image.

14. The medical device according to claim 1, wherein the processor is further configured to:
acquire a blood vessel recognition image, and
generate the display image by superimposing a blood vessel region specified from the blood vessel recognition image on a white light image.

15. The medical device according to claim 14, wherein
the blood vessel recognition image is an image acquired by narrow band light determined based on an absorbance of blood.

16. The medical device according to claim 15, wherein
the blood vessel recognition image is a narrow band light observation image.

17. The medical device according to claim 15, wherein
the blood vessel recognition image is a red light observation image.

18. A medical system comprising:
a light source device including a light source configured to emit excitation light for exciting an advanced glycation end-product generated by subjecting a biological tissue to a thermal treatment;
an imaging device including an imaging element configured to generate an imaging signal by capturing fluorescence emitted by the excitation light; and
a medical device comprising a processor comprising hardware, the processor being configured to:
acquire, from the imaging element, a display image in which a blood vessel region of a blood vessel in a subject is specified and a fluorescence image overlapping at least a part of a visual field region of the display image;
specify thermal denaturation information in the blood vessel region based on the display image and the fluorescence image; and
output the thermal denaturation information.

19. An operation method of a medical device comprising a processor, the operation method causing the processor to execute:
acquiring a display image in which a blood vessel region of a blood vessel in a subject is specified and a fluorescence image overlapping at least a part of a visual field region of the display image;
specifying thermal denaturation information in the blood vessel region based on the display image and the fluorescence image; and
outputting the thermal denaturation information.

20. A non-transitory computer-readable recording medium with an executable program stored thereon, the program causing a processor of a medical device driven to execute:
acquiring a display image in which a blood vessel region of a blood vessel in a subject is specified and a fluorescence image overlapping at least a part of a visual field region of the display image;
specifying thermal denaturation information in the blood vessel region based on the display image and the fluorescence image; and
outputting the thermal denaturation information.
